(19) United States
(12) Patent Application Publication
(10) Pub. No.: US 2023/0338726 A1
(43) Pub. Date: Oct. 26, 2023

(54) PERCUTANEOUS CIRCULATORY SUPPORT DEVICE INCLUDING CURVED PROXIMAL CATHETER

(71) Applicant: Boston Scientific Scimed Inc., Maple Grove, MN (US)

(72) Inventors: Corey N. Meyer, Zemmerman, MN (US); Ian Meredith, Boston, MA (US)

(21) Appl. No.: 18/138,585

(22) Filed: Apr. 24, 2023

Related U.S. Application Data
(60) Provisional application No. 63/334,427, filed on Apr. 25, 2022.

(51) Int. Cl. A61M 60/13; A61M 60/216; A61M 60/857; A61M 60/174

(52) U.S. Cl. CPC A61M 60/13; A61M 60/216; A61M 60/857; A61M 60/174; A61M 25/0152

(57) ABSTRACT

A percutaneous circulatory support device includes an impeller portion having a proximal end portion and an impeller being rotatable to cause blood to flow through the percutaneous circulatory support device. A catheter is coupled to the proximal end portion, and the catheter includes a preformed curve.

### CROSS-REFERENCE TO RELATED APPLICATIONS

This application claims priority to U.S. Provisional Application No. 63/334,427, filed Apr. 25, 2022, which is herein incorporated by reference in its entirety.

### TECHNICAL FIELD

The present disclosure relates to percutaneous circulatory support devices. More specifically, the present disclosure relates to percutaneous circulatory support devices including curved proximal catheters.

### BACKGROUND

Percutaneous circulatory support devices such as blood pumps can provide transient support for up to approximately several weeks in patients with compromised heart function or cardiac output. Several components of such devices, including a distal cannula and a proximal catheter, are typically relatively flexible to facilitate navigating the vasculature of a patient. However, after being properly positioned within a patient, such components can contribute to device instability.

### SUMMARY

In an Example 1, a percutaneous circulatory support device includes an impeller portion having a proximal end portion and an impeller being rotatable to cause blood to flow through the percutaneous circulatory support device. A catheter is coupled to the proximal end portion, and the catheter includes a preformed curve having a radius in a range of 2.00 inches to 0.25 inches.

In an Example 2, the percutaneous circulatory support device of Example 1, wherein the radius of the preformed curve is in a range of 1.50 inches to 0.90 inches.

In an Example 3, the percutaneous circulatory support device of Example 1, wherein the radius of the preformed curve is in a range of 1.40 inches to 1.00 inches.

In an Example 4, the percutaneous circulatory support device of any of Examples 1-3, wherein the preformed curve is offset from the proximal end portion of the impeller portion by a distance in a range of 1.00 inches to 0.05 inches.

In an Example 5, the percutaneous circulatory support device of any of Examples 1-3, wherein the preformed curve is offset from the proximal end portion of the impeller portion by a distance in a range of 0.65 inches to 0.05 inches.

In an Example 6, the percutaneous circulatory support device of any of Examples 1-3, wherein the preformed curve is offset from the proximal end portion of the impeller portion by a distance in a range of 0.55 inches to 0.15 inches.

In an Example 7, the percutaneous circulatory support device of any of Examples 1-6, wherein the preformed curve has an arc length in a range of 1.88 inches to 0.63 inches.

In an Example 8, the percutaneous circulatory support device of any of Examples 1-6, wherein the preformed curve has an arc length in a range of 1.70 inches to 1.10 inches.

In an Example 9, the percutaneous circulatory support device of any of Examples 1-6, wherein the preformed curve has an arc length in a range of 1.60 inches to 1.20 inches.

In an Example 10, the percutaneous circulatory support device of any of Examples 1-9, wherein the impeller portion further includes a distal end portion, further including a cannula coupled to the distal end portion, wherein the preformed curve is a preformed catheter curve, and the cannula includes a preformed cannula curve.

In an Example 11, the percutaneous circulatory support device of Example 10, wherein the preformed catheter curve and the preformed cannula curve are disposed in a common plane.

In an Example 12, a percutaneous circulatory support device includes an impeller portion including a proximal end portion and an impeller being rotatable to cause blood to flow through the percutaneous circulatory support device; and a catheter coupled to the proximal end portion, the catheter including a preformed curve having an arc length in a range of 1.88 inches to 0.63 inches and being offset from the proximal end portion by a distance in a range of 1.00 inches to 0.05 inches.

In an Example 13, the percutaneous circulatory support device of Example 12, wherein the preformed curve has a radius in a range of 2.00 inches to 0.25 inches.

In an Example 14, the percutaneous circulatory support device of any of Examples 12-13, wherein the impeller portion further includes a distal end portion, further including a cannula coupled to the distal end portion, wherein the preformed curve is a preformed catheter curve, and the cannula includes a preformed cannula curve.

In an Example 15, the percutaneous circulatory support device of Example 14, wherein the preformed catheter curve and the preformed cannula curve are disposed in a common plane.

In an Example 16, a percutaneous circulatory support device includes an impeller portion having a distal end portion, a proximal end portion, and an impeller being rotatable to cause blood to flow through the percutaneous circulatory support device. A cannula is coupled to the distal end portion, and a catheter is coupled to the proximal end portion. The catheter has a preformed curve having a radius in a range of 2.00 inches to 0.25 inches.

In an Example 17, the percutaneous circulatory support device of Example 16, wherein the radius of the preformed curve is in a range of 1.50 inches to 0.90 inches.

In an Example 18, the percutaneous circulatory support device of Example 16, wherein the radius of the preformed curve is in a range of 1.40 inches to 1.00 inches.

In an Example 19, the percutaneous circulatory support device of Example 16, wherein the preformed curve is offset from the proximal end portion of the impeller portion by a distance in a range of 1.00 inches to 0.05 inches.

In an Example 20, the percutaneous circulatory support device of Example 16, wherein the preformed curve is offset from the proximal end portion of the impeller portion by a distance in a range of 0.65 inches to 0.05 inches.

In an Example 21, the percutaneous circulatory support device of Example 16, wherein the preformed curve is offset from the proximal end portion of the impeller portion by a distance in a range of 0.55 inches to 0.15 inches.

In an Example 22, the percutaneous circulatory support device of Example 16, wherein the preformed curve has an arc length in a range of 1.88 inches to 0.63 inches.

In an Example 23, the percutaneous circulatory support device of Example 16, wherein the preformed curve has an arc length in a range of 1.70 inches to 1.10 inches.

In an Example 24, the percutaneous circulatory support device of Example 16, wherein the preformed curve has an arc length in a range of 1.60 inches to 1.20 inches.

In an Example 25, the percutaneous circulatory support device of Example 16, wherein the preformed curve is a preformed catheter curve, and the cannula includes a preformed cannula curve.

In an Example 26, the percutaneous circulatory support device of Example 25, wherein the preformed catheter curve and the preformed cannula curve are disposed in a common plane.

In an Example 27, a percutaneous circulatory support device includes an impeller portion having a distal end portion, a proximal end portion, and an impeller being rotatable to cause blood to flow through the percutaneous circulatory support device. A cannula is coupled to the distal end portion, and a catheter is coupled to the proximal end portion. The catheter includes a preformed curve having an arc length in a range of 1.88 inches to 0.63 inches and being offset from the proximal end portion by a distance in a range of 1.00 inches to 0.05 inches.

In an Example 28, the percutaneous circulatory support device of Example 27, wherein the preformed curve has a radius in a range of 2.00 inches to 0.25 inches.

In an Example 29, the percutaneous circulatory support device of Example 27, wherein the preformed curve is a preformed catheter curve, and the cannula includes a preformed cannula curve.

In an Example 30, the percutaneous circulatory support device of Example 29, wherein the preformed catheter curve and the preformed cannula curve are disposed in a common plane.

In an Example 31, a method of manufacturing a percutaneous circulatory support device includes: providing an impeller portion including a distal end portion, a proximal end portion, and a rotatable impeller; coupling a cannula to the distal end portion; and coupling a catheter to the proximal end portion, the catheter including a preformed curve having a radius in a range of 2.00 inches to 0.25 inches.

In an Example 32, the method of Example 31, wherein the preformed curve has an arc length in a range of 1.88 inches to 0.63 inches.

In an Example 33, the method of Example 31, wherein the preformed curve is offset from the proximal end portion of the impeller portion by a distance in a range of 1.00 inches to 0.05 inches.

In an Example 34, the method of Example 31, wherein the preformed curve is a preformed catheter curve, and the cannula includes a preformed cannula curve.

In an Example 35, the method of Example 31, wherein the preformed catheter curve and the preformed cannula curve are disposed in a common plane.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a partial side view of an illustrative percutaneous circulatory support device (also referred to herein, interchangeably, as a “blood pump”) positioned in the aorta and the left ventricle of a patient, in accordance with embodiments of the subject matter disclosed herein.

FIG. 2 is another partial side view of the percutaneous circulatory support device of FIG. 1.

FIG. 3 is a detail side view of the percutaneous circulatory support device within line 3-3 of FIG. 2.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

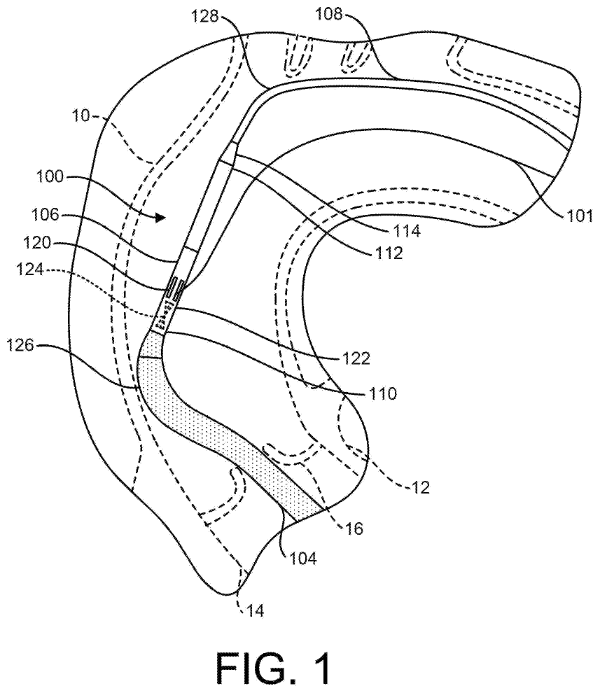

FIG. 1 depicts a portion of an illustrative percutaneous mechanical circulatory support device 100 (also referred to herein, interchangeably, as a “blood pump”), and its relative position in a human aorta 10 and heart 12, more specifically the left ventricle 14, in accordance with embodiments of the subject matter disclosed herein. The blood pump 100 may be delivered percutaneously by passing through the aorta 10, and positioned with the heart 12 with respect to the left ventricle 14 and the aortic valve 16, as shown in FIG. 1. In some embodiments and as illustrated, the blood pump 100 may be delivered using a guidewire 101.

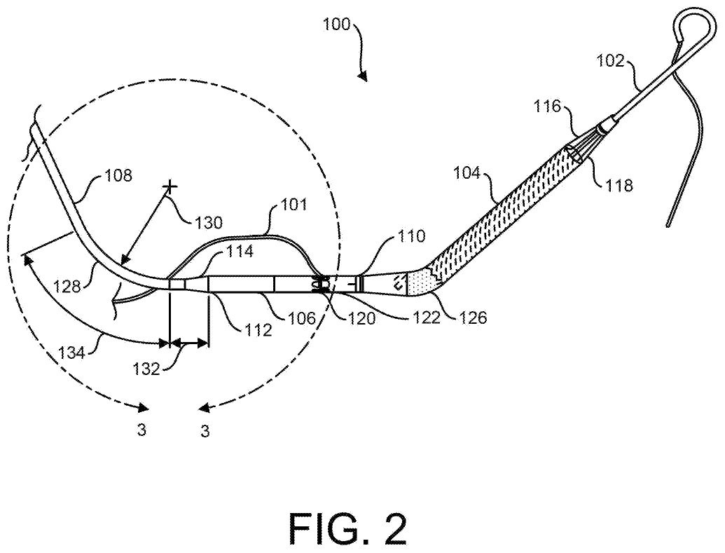

With continued reference to FIG. 1 and additional reference to FIG. 2, the blood pump 100 generally includes a distal extension 102 (FIG. 2), a cannula 104, an impeller portion 106, and a proximal catheter 108. The cannula 104 couples to a distal end portion 110 of the impeller portion 106, and the catheter 108 couples to a proximal end portion 112 of the impeller portion 106, for example, via a tapering connector 114. The cannula 104 and the catheter may have flexible constructions to facilitate delivery of the blood pump 100. The cannula 104 includes one or more blood inlets 116 (FIG. 2) located on a distal portion 118 thereof, and one or more blood outlets 120 are located on a housing 122 of the impeller portion 106. The housing 122 carries an impeller 124 (FIG. 1), and the impeller 124 rotates relative to the housing 122 to cause blood to flow into the inlets 116, through the housing 122, and out of the outlets 120. During operation and as shown in FIG. 1, the blood pump 100 is positioned within the heart 12 such that the inlets 116 (FIG. 2) are positioned in the left ventricle 14 and the outlets 120 are positioned in the aorta 10. As a result, rotation of the impeller 124 relative to the housing 122 causes blood to flow from the left ventricle 14, through the cannula 104 and the impeller portion 106, and into the aorta 10.

With continued reference to FIGS. 1 and 2, the cannula 104 and the catheter 108 both include preformed curves (as used herein, the term “preformed” and variations thereof meaning that a feature is present prior to entering the vasculature of a patient). More specifically, the cannula 104 includes a bent longitudinal axis that provides a first preformed curve 126 (also referred to as a preformed cannula curve 126) and the catheter 108 includes a bent longitudinal axis that provides a second preformed curve 128 (also referred to as a preformed catheter curve 128). The preformed curves 126, 128 advantageously facilitate applying relatively low forces to the aortic valve 16. Similarly, the preformed catheter curve 128 reduces or eliminates a tendency for the catheter 108 to straighten in the aorta 10. As a result, the preformed curves 126, 128 facilitate device stability and proper positioning of the blood pump 100, including the proximal catheter 108 and cannula 104, within the patient anatomy. In some embodiments and as illustrated, the preformed catheter curve 128 and the preformed cannula curve 126 are disposed in a common plane.

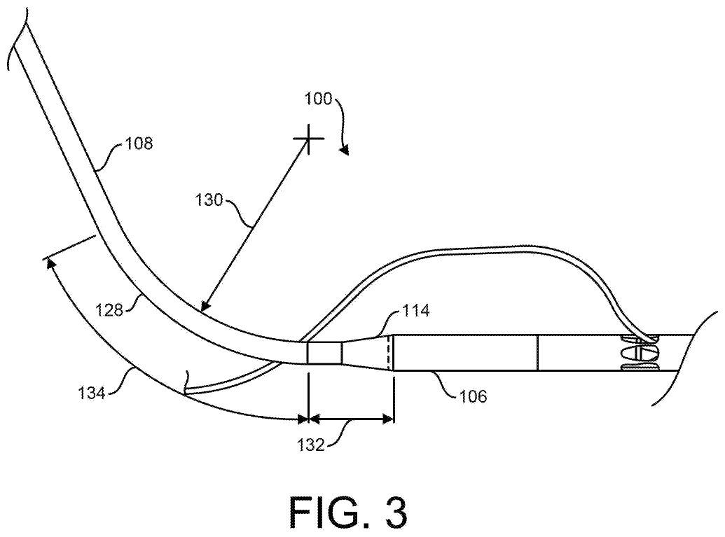

The preformed catheter curve 128 may have various dimensions. For example and with reference to FIGS. 2 and 3, the preformed catheter curve 128 may have a radius 130 in a range of 1.50 inches to 0.90 inches, a range of 1.40 inches to 1.00 inches, or a range of 1.30 inches to 1.10 inches. The preformed catheter curve 128 may be offset from the proximal end portion 112 of the impeller portion 106 by a distance 132 in a range of 0.65 inches to 0.05 inches, a range of 0.55 inches to 0.15 inches, or a range of 0.45 inches to 0.25 inches. The preformed catheter curve 128 may comprise an arc in a range of 80 degrees to 53 degrees, a range of 76 degrees to 57 degrees, or a range of 72 degrees to 62 degrees (that is, an arc length 134 in a range of 1.70 inches to 1.10 inches, a range of 1.60 inches to 1.20 inches, or a range of 1.50 inches to 1.30 inches).

As another example, the preformed catheter curve 128 may have a radius 130 in a range of 2.00 inches to 0.25 inches. The preformed catheter curve 128 may be offset from the proximal end portion 112 of the impeller portion 106 by a distance 132 in a range of 1.00 inches to 0.05 inches. The preformed catheter curve 128 may comprise an arc of 90 degrees to 30 degrees (that is, an arc length 134 in a range of 1.88 inches to 0.63 inches).

A method of manufacturing a blood pump in accordance with embodiments of the subject matter disclosed herein may be generally as follows. The method describes features of the blood pump 100, although it is understood that any blood pump contemplated herein could be used in a similar manner. Initially, the impeller portion 106, the cannula 104, and the catheter 108 are provided. Next, the cannula 104 is coupled to the distal end portion 110 of the impeller portion 106 (via welding, bonding, or the like) and the catheter 108 is coupled to the proximal end portion 112 of the impeller portion 106. The curve 128 is then formed in the catheter 108, for example, by applying heated air to the catheter 108 and bending the catheter 108 on a mandrel (not shown). Alternatively, the curve 128 may be formed in the catheter 108 prior to coupling the catheter 108 to the impeller portion 106.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A percutaneous circulatory support device, comprising:
an impeller portion comprising;
a distal end portion;
a proximal end portion; and
an impeller being rotatable to cause blood to flow through the percutaneous circulatory support device;
a cannula coupled to the distal end portion; and
a catheter coupled to the proximal end portion, the catheter comprising a preformed curve having a radius in a range of 2.00 inches to 0.25 inches.

2. The percutaneous circulatory support device of claim 1, wherein the radius of the preformed curve is in a range of 1.50 inches to 0.90 inches.

3. The percutaneous circulatory support device of claim 1, wherein the radius of the preformed curve is in a range of 1.40 inches to 1.00 inches.

4. The percutaneous circulatory support device of claim 1, wherein the preformed curve is offset from the proximal end portion of the impeller portion by a distance in a range of 1.00 inches to 0.05 inches.

5. The percutaneous circulatory support device of claim 1, wherein the preformed curve is offset from the proximal end portion of the impeller portion by a distance in a range of 0.65 inches to 0.05 inches.

6. The percutaneous circulatory support device of claim 1, wherein the preformed curve is offset from the proximal end portion of the impeller portion by a distance in a range of 0.55 inches to 0.15 inches.

7. The percutaneous circulatory support device of claim 1, wherein the preformed curve comprises an arc length in a range of 1.88 inches to 0.63 inches.

8. The percutaneous circulatory support device of claim 1, wherein the preformed curve comprises an arc length in a range of 1.70 inches to 1.10 inches.

9. The percutaneous circulatory support device of claim 1, wherein the preformed curve comprises an arc length in a range of 1.60 inches to 1.20 inches.

10. The percutaneous circulatory support device of claim 1, wherein the preformed curve is a preformed catheter curve, and the cannula comprises a preformed cannula curve.

11. The percutaneous circulatory support device of claim 10, wherein the preformed catheter curve and the preformed cannula curve are disposed in a common plane.

12. A percutaneous circulatory support device, comprising:
an impeller portion comprising:
a distal end portion;
a proximal end portion;
an impeller being rotatable to cause blood to flow through the percutaneous circulatory support device;
a cannula coupled to the distal end portion; and
a catheter coupled to the proximal end portion, the catheter comprising a preformed curve having an arc length in a range of 1.88 inches to 0.63 inches and being offset from the proximal end portion by a distance in a range of 1.00 inches to 0.05 inches.

13. The percutaneous circulatory support device of claim 12, wherein the preformed curve comprises a radius in a range of 2.00 inches to 0.25 inches.

14. The percutaneous circulatory support device of claim 12, wherein the preformed curve is a preformed catheter curve, and the cannula comprises a preformed cannula curve.

15. The percutaneous circulatory support device of claim 14, wherein the preformed catheter curve and the preformed cannula curve are disposed in a common plane.

16. A method of manufacturing a percutaneous circulatory support device, the method comprising:
providing an impeller portion comprising a distal end portion, a proximal end portion, and a rotatable impeller;
coupling a cannula to the distal end portion; and
coupling a catheter to the proximal end portion, the catheter comprising a preformed curve having a radius in a range of 2.00 inches to 0.25 inches.

17. The method of claim 16, wherein the preformed curve comprises an arc length in a range of 1.88 inches to 0.63 inches.

18. The method of claim 16, wherein the preformed curve is offset from the proximal end portion of the impeller portion by a distance in a range of 1.00 inches to 0.05 inches.

19. The method of claim 16, wherein the preformed curve is a preformed catheter curve, and the cannula comprises a preformed cannula curve.

20. The method of claim 16, wherein the preformed catheter curve and the preformed cannula curve are disposed in a common plane.
